(19) United States
(12) Patent Application Publication
(10) Pub. No.: US 2007/0054908 A1
(43) Pub. Date: Mar. 8, 2007

(54) Novel pharmaceutical compositions and methods for treating tumors, including drug-resistant tumors

(76) Inventors: Zhongyun Dong, Cincinnati, OH (US); Shan Lu, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 11/515,052

(22) Filed: Sep. 1, 2006

Related U.S. Application Data
(60) Provisional application No. 60/714,123, filed on Sep. 2, 2005.

(51) Int. Cl. A61K 31/5375; A61K 31/445

(52) U.S. Cl. 514/237.5; 514/317

(57) ABSTRACT

Pharmaceutical compositions comprising a chemotherapeutic agent comprising one of the previously unknown antineoplastic agents, DL-001 or DL-002 and methods for inhibiting tumor cell proliferation, inducing tumor cell death by apoptosis and inhibiting growth of a drug resistant tumor comprising administration of the inventive compositions are provided. Successfully treated tumors include epithelial cell-derived malignancies and a broad spectrum of tumors, including drug resistant tumors. In particular, the inventive compositions retain efficacy in tumors exhibiting multiple drug resistance.

### RELATED APPLICATION

This application claims priority under 35 U.S.C. §119 to U.S. Provisional Application Ser. No. 60/714,123, filed on Sep. 2, 2005.

### FIELD OF THE INVENTION

The invention relates to novel pharmaceutical compositions comprising small molecule drug agents, 4-Methoxy-phenyl)-(5-nitro-2-piperidin-1-yl-phenyl)-methanone, and 4-Methoxy-phenyl)-2-morpholin-4-yl-5-nitro-phenyl)-methanone. The agents and compositions are useful for inducing the death of epithelial cell-derived malignant cells from a broad spectrum of tumorous cells. The invention further relates to methods of treating tumors, including drug resistant tumors.

### BACKGROUND OF THE INVENTION

Several cancers have proven particularly resistant to currently available chemotherapeutic interventions. Other cancers are prone to develop multiple drug resistance, and there are few chemotherapeutic agents capable of thwarting this resistance. For example, prostate cancer, the most common cancer and the second most common cause of cancer death among men in the United States, is particularly problematic in this regard. It is expected that 232,090 men will be diagnosed of and 30,350 men will die from this disease in 2005. As detection techniques improve, increasing numbers of patients are being diagnosed with localized disease, whereas the number of patients diagnosed with disseminated diseases is on the decline. Metastasis in many patients, however, still occurs prior to the initial diagnosis and an unfortunate result is that eradication of primary tumors by either surgery or radiation therapy is not curative. Hormonal manipulations by androgen ablation and anti-androgen agents typically only slow the progression of advanced prostate cancer by an average of less than 18 months.

Systemic chemotherapies employing specific small molecule compounds can potentially destroy both primary tumors and micrometastases. However, prostate cancer has proven substantially insensitive to the chemotherapeutic agents currently available. Therefore, there is an urgent need for development of chemotherapeutic compounds effective for treating prostate cancer, and other similarly resistant cancers.

According to currently popular strategies, most researchers seeking cancer therapy drugs focus on development of agents that target specific signaling molecules or pathways necessary for cell survival and migration, seeking to destroy only tumor cells. However, malignant and normal cells typically use the same pathways for growth and migration, hence, with a few exceptions, most drugs developed according to current protocols kill both tumor and normal cells similarly. Therefore, the present inventors elected to investigate potential drug agents using traditional cytotoxicity-based screening strategies.

Certain cancer chemotherapies may fail because of resistance of tumors to multiple drug therapies. Commonly, a tumor may become resistant to the anti-tumor agent used in a previous treatment. The therapeutic effects of these agents are then eliminated in subsequent treatment protocols. More problematic is that recurrent cancers are resistant not only to the cancer suppressants used in previous treatments, but also demonstrate resistance to other antitumor agents, unrelated to the agent used previously either by chemical structure or by mechanism of action. These phenomenon are collectively referred to as multiple drug resistance and contribute widely to cancer treatment failures in the clinic. Hence, there is an urgent need for chemotherapeutic agents which avoid the resistance engendered by prior treatments using commonly employed chemotherapeutic agents.

### SUMMARY OF THE INVENTION

Accordingly, the present invention provides novel pharmaceutical compositions comprising compounds exhibiting cytotoxic efficacy against tumor cells derived from a broad range of tumors, and methods for treating tumors in subjects suffering from such tumors, including methods for treating drug resistant tumors. The mechanism of cell death is by induction of cell apoptosis, thus there is a minimum of toxic side-effects typically seen with agents that operate via mechanisms that cause cell death via cell necrosis.

One embodiment of the invention provides a pharmaceutical composition comprising: a chemotherapeutic agent comprising one of the compounds designated herein as DL-001 and DL-002 (see structures below), or a pharmaceutically acceptable salt thereof; and one or more pharmaceutically acceptable excipient.

Another embodiment is directed to methods for inhibiting tumor cell proliferation by administration of the inventive pharmaceutical compositions. A further embodiment provides a method of inducing tumor cell death by apoptosis by administration of the inventive compositions.

In one embodiment, a method of inhibiting growth of a drug resistant tumor is provided. The method comprises administering a pharmaceutical composition comprising DL-001 or DL-002, or a pharmaceutically acceptable salt thereof, wherein the pharmaceutical composition is administered at a dose therapeutically effective to induce tumor cell death by apoptosis.

These and other embodiments will be more fully appreciated by reference to the Figures and Detailed Description of the Invention, as set forth below.

### BRIEF DESCRIPTION OF THE DRAWINGS

FIG. 1 Effects of DL-001 on PC-3 tumor growth in the prostate of nude mice.

FIG. 2 Effects of DL-001 on cell cycle and gene expression.

### DETAILED DESCRIPTION OF THE INVENTION

A library of 10,000 compounds selected by the inventors for its molecular property characteristic profile, was purchased from Chembridge Corporation. The library was selected for molecular characteristics considered desirable in small molecule potential drug agents, including but not limited to diversity, low molecular weight (250-450), low polar surface area, rotatable bonds, hydrogen donor behavior, and hydrogen acceptor value ranges. Using a high throughput cytotoxic assay developed in part by the present inventors, and disclosed in Dong Z. et al. “In Vitro Model For Intrinsic Drug Resistance: Effects Of Protein Kinase C Activators On the Chemosensitivity Of Cultured Human Colon Cancer Cells” Mol Pharmacol 1991;39(4):563-9, the disclosure of which is fully incorporated herein by this reference, two compounds having nearly the same structure except for a single carbon to oxygen ring element substitution, were identified as particularly promising cytotoxic drug candidates and thereafter subjected to extensive pharmaceutical evaluation. These two compounds, (4-Methoxy-phenyl)-(5-nitro-2-piperidin-1-yl-phenyl)-methanone and (4-Methoxy-phenyl)-(2-morpholin-4-yl-5-nitro-phenyl)-methanone are named DL-001 and DL-002, respectively, and are set forth structurally, below.

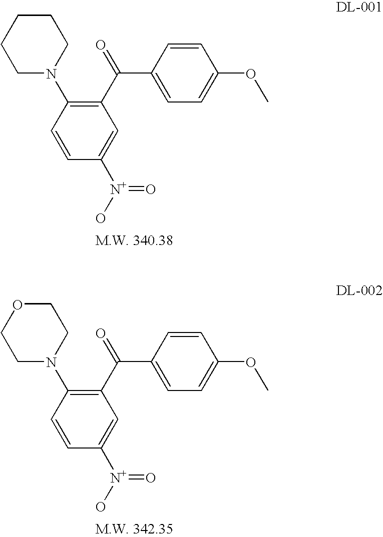

The two compounds, DL-001 and DL-002, demonstrate chemotherapeutic efficacy in inhibiting proliferative cells such as neoplastic tumor cells derived from a broad range of tumors, while exhibiting insignificant toxic side effects. The compounds, in particular DL-001, exhibit a remarkable retention of cytotoxic efficacy in drug-resistant tumors, including tumors with inherent resistance and tumors exhibiting acquired multiple drug resistance. Notably, the compounds retain cytotoxic efficacy with respect to tumors that are substantially resistant to antineoplastic phlylogenic agents such as taxol and vincristine. Interestingly, it appears that DL-001 and DL-002 act via a similar mechanism to these known agents, by inducing cell death via apoptosis. Antineoplastic phylogenic agents derive from higher plants and have demonstrable cytostatic or antineoplasitc activity. Two popular and commonly employed phylogenic agents include Taxol, isolated from the stem bark of the western yew, Taxus brevifolia [Wani et al., J. Amer. Chem. Soc., 93: 2325-2327 (1971)], and vincristine, an alkaloid isolated from Vinca Rosea (Merck, 11^{th} ed.). These particular phylogenic agents are set forth herein for comparative and illustrative purposes, and it is contemplated that the present compounds will avoid drug resistance acquired due to treatment regimens employing other antineoplastic agents as well.

One embodiment of the invention is directed to a pharmaceutical composition comprising: a chemotherapeutic agent comprising DL-001, DL-002, or a pharmaceutically acceptable salt thereof; and one or more pharmaceutically acceptable excipients. In a more specific embodiment, the chemotherapeutic agent comprises a therapeutically effective amount of DL-001, and in another specific embodiment, the chemotherapeutic agent comprises a therapeutically effective amount of DL-002.

As noted, compounds of DL-001 and DL-002 may be administered per se or in the form of a pharmaceutically acceptable salt. When used in pharmaceutical compositions, the reference to salts of either compound as “pharmaceutically acceptable” is understood as both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts include, but are not limited to, those prepared from the following acids: hydrochloric, hydrobromic, sulphuric, nitric, phosphoric, maleic, salicylic, p-toluenesulfonic, tartaric, citric, methanesulphonic, formic, malonic, succinic, naphthalene-2-sulphonic and benzenesulphonic. Also, pharmaceutically acceptable salts can be prepared as alkaline metal or alkaline earth salts, such as sodium, potassium or calcium salts of the carboxylic acid group. Thus, the present invention provides pharmaceutical compositions, both for veterinary and for human applications, which comprise the chemotherapeutic agents, or the pharmaceutically acceptable salt thereof, and one or more pharmaceutically acceptable carriers. A carrier is pharmaceutically acceptable if it is compatible with the other ingredients of the formulation and not unduly deleterious to the recipient thereof. The compounds of the present disclosure may be hydrated or non-hydrated.

Subjects to be treated according to the present invention include both human and animal (e.g., mouse, dog, cat, cow, horse) subjects, specifically mammalian subjects, and more specifically, human subjects.

As is common practice in the pharmaceutical arts, a physician will determine the actual dosage which is most suitable for a particular individual subject and the a therapeutically effective dose typically varies with the age, weight and response pattern of the particular patient. The dosages disclosed herein, therefore, are intended to illustrate a general case and it is understood that circumstances will exist whereby higher or lower dosage ranges are warranted.

The pharmaceutical compositions of the present invention may be formulated for parenteral, mucosal, intramuscular, intravenous, subcutaneous, intraocular or transdermal administration. Depending upon the need, the active agent may be administered at a dose of from about 1 to 500 mg/kg body weight, such as from 20 to 300 mg/kg, and, in further specific embodiments, from about 100 to about 200 mg/kg body weight. Since dose may be determined on a per-case basis, for purposes of the present invention, although only range endpoints are expressly specified, the contemplated scope includes potential range endpoints falling anywhere within the expressed ranges, between and including the expressed endpoints.

Another embodiment is directed to a method for inhibiting tumor cell proliferation. The method comprises administration of the inventive pharmaceutical composition comprising either of DL-001 or DL-002 as the active chemotherapeutic agents. In a specific embodiment, the chemotherapeutic agent is DL-001. The inventors conducted successful studies on several representative epithelial cell-derived malignancies, and, without intending to be bound by theory, discovered that the agents operate via an apoptotic mechanism that is applicable to all epithelial cell-derived malignancies. In a more specific embodiment, the tumor cell derives from a tumor of the type selected from the group consisting of breast cancer, cancers of the central nervous system, colon cancer, kidney cancer, liver cancer, lung cancer, prostate cancer, melanoma, urinary bladder cancer, sarcoma, fibrosarcoma, leukemia and lymphoma. The tumor may be a primary tumor, or, of particular note, the present compositions and methods have particular efficacy in treating tumors where the tumor is a metastasis from a primary tumor. In very specific embodiments the tumor cell derives from prostate or colon cancer, and according to even more specific embodiments, the tumor cell derives from prostate cancer. Further, the tumor cell may derive from a solid tumor, a non-solid tumor or a lymphoma.

According to another specific embodiment, the tumor cell is a malignant skin cell. The malignant skin cell may be a basal cell carcinoma, a squamous cell carcinoma, a melanoma, a sarcoma, and a lymphoma.

The present invention also provides a method of inducing tumor cell death by apoptosis. The method comprises administration of one or more embodiments of the inventive pharmaceutical compositions. Cytotoxicity or cell death may occur via either necrosis or apoptosis. Necrosis is typically the result of physical or chemical injury, while apoptosis is a cellular response to a specific stimuli, e.g., a cell surface-generated signal. Necrosis may involve the destruction of cytoplasmic organelles and a loss of plasma membrane integrity, whereas cells undergoing apoptosis exhibit cell shrinkage, membrane blebbing, chromatin condensation and fragmentation. A substantive difference involves the presence of an inflammatory response in the case of cell necrosis, which is typically avoided in the case of apoptosis, such that cell death via apoptosis minimizes damage to surrounding cells and tissue.

According to certain embodiments, the inventive compositions may be administered as an adjuvant therapeutic agent. In particular embodiments, the inventive compositions may be co-administered with at least one other chemotherapeutic agent. This agent may be an antineoplastic agent that inhibits tumor cell proliferation via the same or different mechanisms. According to one embodiment, the inventive method further comprises co-administration of an antineoplastic agent that inhibits tumor cell proliferation via a mechanism other than cell apoptosis. A “co-administered agent may be administered before, after or simultaneously with the compounds according to the invention. Co-administered, according to the present invention, is understood to mean as part of a single treatment regimen, and does not necessarily imply cotemporaneous administration.

The agents of the present invention may be formulated into compositions that are administered in a single controlled release form once per day, or from 1 to 4 times per day. The specific dose level and frequency of dosage for any particular patient will depend upon a variety of factors personal to the patient, including age, body weight, general health, sex, diet, mode and time of administration, rate of excretion, additional treatment regimens, and the severity of the particular condition. Most particularly, the dosage may vary with co-administration of other chemotherapeutic agents or when administration is subsequent to the development of resistance to other chemotherapeutic agents.

Various routes of administration are contemplated as being suitably within the scope of the present invention, including but not limited to mucosal, nasal, oral, parenteral, gastrointestinal, topical, or sublingual routes. A mucosal route may include, for example, administration as a nasal spray or aerosol for inhalation. A parenteral route may include where delivery is by an injectable form, for example, by an intravenous, intramuscular or subcutaneous route. The therapeutic compounds described herein may be formulated into compositions for particular routes of administration utilizing conventional methods. For example, DL-001 or DL-002 may be administered as part of a chemotherapeutic cocktail typically comprising an aqueous saline vehicle, or the pharmaceutical compositions may be formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, a conventional filler, and a tableting agent. The pharmaceutical composition may be administered in a manner which allows direct contact of the surface of the tumor cell. The compound may bind to, e.g., a cell surface moiety to initiate an intracellular signal transduction cascade culminating with cell death by apoptosis. One embodiment of the invention provides a cell growth inhibitor for inhibiting growth of a vascular endothelial cell, comprising a pharmacologically acceptable salt of DL-001 or DL-002. Alternatively, controlled release of the compound may be achieved by implanting a drug loaded matrix in direct contact or adjacent to the tumor site.

Thus, for pharmaceutical administration, the chemotherapeutic agents of the present invention can be formulated in any suitable manner utilizing conventional pharmaceutical formulating techniques and pharmaceutical carriers, adjuvants, excipients, diluents etc.

Approximate therapeutically effective dose rates may be in the range from 5 to 5000 mg/day, for example, from 1000 to 3000 mg/day or from 1500 to 2500 mg/day depending on activity, age and gender of a patient of average (70 Kg) bodyweight. For oral administration unit doses may be formulated in tablets, capsules, solution or suspension containing from 500 to 5000 mg of compound per unit dose. Alternatively, according to one embodiment, the compounds will be formulated for parenteral administration in a suitable parenterally administrable carrier to provide a single daily dosage rate in the range 500 to 5000 mg, more specifically, 1000 to 3000, and even more specifically 1500 to 2500 mg. Such effective daily doses will, however, vary depending on the gender and bodyweight of the patient, such variations being within the skill and judgment of the ordinary physician. In addition, due to the relatively low observed toxicity of DL-001 and DL-002, higher doses may be administered without deleterious side effects.

It is noted that the compounds of the present invention may exist in different isomeric forms and the invention includes all “isomers,” understood to include all possible stereoisomers, geometric isomers, diastereoisomers, enantiomers, anomers and optical isomers of the compounds as set forth and described herein and in the claims. Mixtures of isomers can be separated into individual isomers according to methods which are known per se, e.g. fractional crystallization, adsorption chromatography or other suitable separation processes. Resulting racemates can be separated into antipodes in the usual manner after introduction of suitable salt-forming groupings, e.g. by forming a mixture of diastereosiomeric salts with optically active salt-forming agents, separating the mixture into diastereomeric salts and converting the separated salts into the free compounds. The possible enantiomeric forms may also be separated by fractionation through chiral high pressure liquid chromatography columns.

Certain aspects of the invention may be more clearly understood by reference to the following examples. However, the examples are provided for illustrative purposes only and should not be construed as limiting the full scope of the invention as disclosed and claimed herein.

### EXAMPLE 1

The following example illustrates in vitro evaluation of cytotoxic efficacy for DL-001 and DL-002.

To evaluate the cytotoxic activity of the two compounds, human prostate cancer cells (PC-3, LNCaP, and DU145) were plated into 96-well plate and incubated for 96 hr with various concentrations of desired drugs. During the last 2 hr, MTT (3-(4,5-dimethylthiazol-2-yl)-2,5-diphenyltetrazolium bromide) was added and the dark-blue formazan formed in live cells was dissolved in dimethyl sulfoxide and the absorbance was measured with a FLUOstar Optima microplate reader (BMG LABTECH Inc., Durham, N.C.) at 570 nm. The IC50 was defined as the concentration of a drug that inhibited cell growth by 50% (see Dong et al, supra, and Dong Z, et al. “Organ-specific modulation of steady-state mdr gene expression and drug resistance in murine colon cancer cells” J. Natl. Cancer Inst 1994;86(12):913-20, both references fully incorporated herein by this reference). Data in Table 1 (below) demonstrates that human prostate cancer cells are susceptible to the cytotoxic activity of DL-001.

TABLE 1
IC50 of DL-001 and DL-002

| Cell lines | DL-001 | DL-002 | Taxol | Vincristine |
|---|---|---|---|---|
| Human cells | | | | |
| PC-3 (p53, del) | 32* | 30 | 4 | 4 |
| LNCaP (p53, wt) | 11 | nd | nd | nd |
| DU145 (p53, mt) | 28 | 30 | 3 | 5 |
| MCF-7 (p53, wt) | 17 | nd | nd | nd |
| MDA435 (p53, mt) | 30 | nd | nd | nd |
| A375 (p53, wt) | 23 | nd | nd | nd |
| Mouse cells | | | | |
| TRAMP (prostate) | 41 | nd | nd | nd |
| 4T1 (breast) | 30 | nd | nd | nd |
| B16 (melanoma) | 26 | nd | nd | nd |
| UV2237 (fibrosarcoma) | 35 | nd | nd | nd |
| CT-26 (colon) | 39 | nd | 73 | 16 |
| CT-26 (colon, MDR) | 39 | nd | 367 | 38 |
| mouse endothelial cells | | | | |
| lung | 45 | nd | nd | nd |
| prostate | 51 | nd | nd | nd |

*IC50 values, nM;
nd = not done.
del = deletion,
wt = wildtype,
mt = mutant;
MDR = multidrug resistant.

In addition, the present inventors discovered that the compounds could also effectively kill human breast (MCF-7 and MDA435) and melanoma (A375) cells, as well as several lines of mouse tumor cells, with similar potencies (Table 1). Moreover, DL-001 could effectively kill endothelial cells derived from mouse lung and prostate (Table 1). Furthermore, it appears that tumor cells (LNCaP, MCF-7, and A375) that harbor the wildtype p53 tumor suppressor gene were relatively more sensitive to DL-001 (Table 1). Notably, DL-001, but not Taxol or Vincristine, killed both parental CT-26 and multi-drug resistant CT-26R500 cells with the same potency (Table 1).

### EXAMPLE 2

The following example illustrates cytotoxicity of DL-001 and DL-002 on prostate tumor cells in the absence of significant toxic side effects.

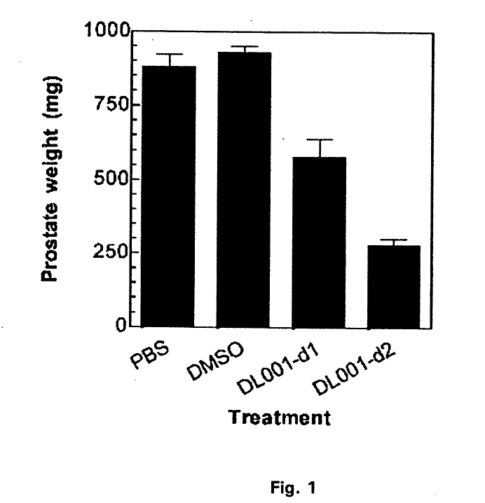

Therapeutic effects of DL-001 and DL-002 on tumor growth in an orthotopic model of PC-3 human prostate cancer cells (See Zhang F, et al. “Blockade of transforming growth factor-beta signaling suppresses progression of androgen-independent human prostate cancer in nude mice” Clin. Cancer Res 2005;11(12):4512-20 and Dong Z, et al. “Suppression of angiogenesis, tumorigenicity, and metastasis by human prostate cancer cells engineered to produce interferon-beta” Cancer Res 1999;59(4):872-9, the disclosure of both articles herein incorporated in their entirely by this reference). was also investigated and demonstrated. PC-3MM2 cells are implanted into the prostate of nude mice. The therapy begins 3 days later, when tumors are established, by daily intraperitoneal injection of DL-001 at 10 mg/kg or 20 mg/kg of body weight or of DMSO (solvent). Experiments are terminated 3 weeks later. Data in FIG. 1 reveal that the therapy with DL-001 inhibited growth of PC-3MM2 tumors in dose-dependent manner. Similar results were obtained in therapy using DL-002 (data not shown). The therapies did not produce significant toxic side effects on mice, based on body weight and H & E-stained paraffin sections of organs (heart, lung, liver, kidney, and spleen.

### EXAMPLE 3

The following example illustrates that DL-001 acts by inducing cell apoptosis.

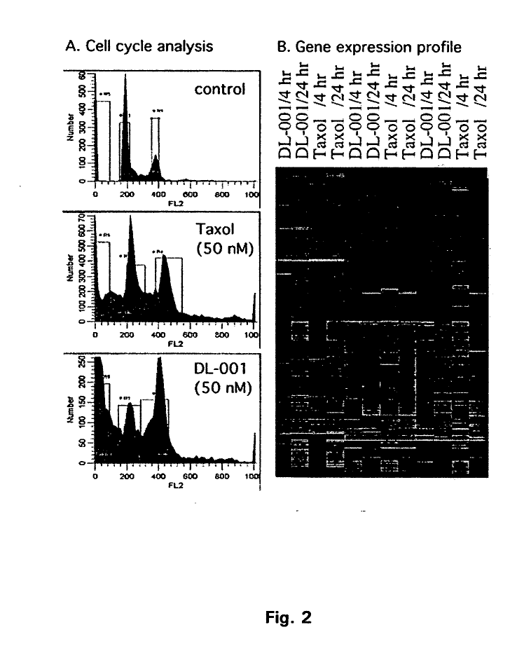

Taxol is known to kill tumor cells by inducing arrest of cells at S/G2 cell cycle and apoptosis. To identify mechanisms by which DL-001 killed tumor cells, we compared effects of DL-001 and Taxol on PC-3MM2 cells. Data in FIG. 3A show that PC-3MM2 cells exposed DL-001 or Taxol were arrested at S/G2 checkpoint of cell cycle and went into apoptosis. Moreover, it is noteworthy that LNCaP cells and PC-3 cells exposed to taxol and DL-001 displayed strikingly similar gene expression patterns revealed by oligo-DNA microassay analysis that covered 16,000 genes (FIG. 2B). To verify the role of DL-001 in inducing apoptosis, the activity of caspase 2,3,6,8 and 9 was analyzed. PC-3MM2 cells are treated with 50 nM of DL-001, taxol, or vincristine for 24 hr. Caspase activities in the treated cells are assessed by using a fluorometric caspase assay kit (Bio Vision, Mountain View, Calif.). As shown in Table 2, the treatment with all three drugs induced activation of caspases 2, 3, and 9. Together with the finding that cells harboring wildtype p53, a DNA damage sensor, were relatively more sensitive to DL-001 (Table 1), the data suggest that DL-001-induced apoptosis is DNA damage and genotoxic stress-related.

TABLE 2
Activation of Caspases

| Caspases | Control | DL-001 | Taxol | Vincristine |
|---|---|---|---|---|
| 2 | 1 | 17* | 26 | 18 |
| 3 | 1 | 22 | 34 | 24 |
| 6 | 1 | 1 | 1 | 1 |
| 8 | 1 | 1 | 1 | 1 |
| 9 | 1 | 4 | 7 | 4 |

*Fold change of caspase activity compared with that in control cells

What is claimed:

1. A pharmaceutical composition comprising: a chemotherapeutic agent comprising DL-001, DL-002, or a pharmaceutically acceptable salt thereof; and one or more pharmaceutically acceptable excipients.

2. The pharmaceutical composition according to claim 1, wherein the chemotherapeutic agent comprises a therapeutically effective amount of DL-001.

3. A method for inhibiting tumor cell proliferation, the method comprising administration of the pharmaceutical composition according to claim 1.

4. A method for inhibiting tumor cell proliferation, the method comprising administration of the pharmaceutical composition according to claim 2.

5. The method according to claim 3, wherein the tumor cell comprises an epithelial cell-derived malignant cell.

6. The method according to claim 3, wherein the tumor cell derives from a tumor of the type selected from the group consisting of breast cancer, cancers of the central nervous system, colon cancer, kidney cancer, liver cancer, lung cancer, prostate cancer, melanoma, urinary bladder cancer, sarcoma, fibrosarcoma, leukemia and lymphoma.

7. The method according to claim 6, wherein the tumor cell derives from prostate cancer.

8. The method according to claim 3, wherein the tumor is a metastasis from a primary tumor.

9. The method according to claim 3, wherein the tumor cell is derived from a solid tumor, a non-solid tumor or a lymphoma.

10. The method according to claim 3, wherein the tumor cell is a malignant skin cell.

11. The method according to claim 10, wherein the malignant skin cell is selected from the group consisting of a basal cell carcinoma, a squamous cell carcinoma, a melanoma, a sarcoma, and a lymphoma.

12. A method of inducing tumor cell death by apoptosis, the method comprising administration of the pharmaceutical composition according to claim 1.

13. The method according to claim 3, further comprising co-administration of an antineoplastic agent that inhibits tumor cell proliferation via a mechanism other than cell apoptosis.

14. A method of inhibiting growth of a drug resistant tumor, the method comprising administering a pharmaceutical composition comprising DL-001 or DL-002, or a pharmaceutically acceptable salt thereof, wherein the pharmaceutical composition is administered at a dose therapeutically effective to induce tumor cell death by apoptosis.

15. The method according to claim 14, wherein the drug resistant tumor expresses a gene encoding multiple drug resistance.

16. The method according to claim 14, wherein the drug resistant tumor manifests resistance to an antineoplastic phylogenic agent.

17. The method according to claim 14, wherein the antineoplastic phylogenic agent comprises Taxol.

18. The method according to claim 14, wherein the antineoplastic phylogenic agent comprises Vincristine.

19. The method according to claim 14, wherein the drug resistant tumor comprises an epithelial cell-derived malignancy.

20. The method according to claim 14, wherein the drug resistant tumor is selected from the group consisting of breast cancer, cancers of the central nervous system, colon cancer, kidney cancer, liver cancer, lung cancer, prostate cancer, melanoma, urinary bladder cancer, sarcoma, fibrosarcoma, leukemia and lymphoma.

21. The method according to claim 20, wherein the drug resistant tumor is colon cancer.

22. The method according to claim 14, wherein the tumor is a solid tumor, a non-solid tumor, or a lymphoma.

23. A cell growth inhibitor for inhibiting growth of a vascular endothelial cell, comprising a pharmacologically acceptable salt of DL-001 or DL-002.
